(19) United States
(12) Patent Application Publication
(10) Pub. No.: US 2025/0295359 A1
(43) Pub. Date: Sep. 25, 2025

(54) INTERVENTION BASED ON DETECTED GAIT KINEMATICS

(71) Applicant: MAGNES AG, Zurich (CH)

(72) Inventors: Barna BECSEK, Zurich (CH); Henrik MAURENBRECHER, Zurich (CH); Georgios CHATZIPIRPIRIDIS, Zurich (CH); Olgac ERGENEMAN, Zurich (CH); Alessandro SCHAER, Zurich (CH)

(21) Appl. No.: 18/869,298

(22) PCT Filed: May 24, 2023
(86) PCT No.: PCT/EP2023/063911

§ 371 Date: Nov. 26, 2024

(30) Foreign Application Priority Data
May 26, 2022 (GB) 2207798.6

(51) Int. Cl. A61B 5/00; A61B 5/11; A61N 1/36; G16H 20/30; G16H 40/67

(52) U.S. Cl. CPC A61B 5/6807; A61B 5/112; A61N 1/36; G16H 20/30; G16H 40/67; A61B 2562/0219; A61B 2562/0247; A61B 2562/0271

(57) ABSTRACT

A system for providing an intervention based on detected gait kinematics for therapy, training, gaming or movement assistance. The system comprises at least one item of footwear incorporating a sensor or sensors, a data processor, and a wireless communication unit, and a remote intervention system configured to provide an intervention for provoking a response by a subject wearing the item of footwear. The sensor or sensors are configured to generate sensor data associated with movement of the subject. The data processor is configured to process the sensor data to generate gait parameter data associated with the subject's gait kinematics, and the wireless communication unit is configured to communicate the gait parameter data to the remote intervention system.

### TECHNICAL FIELD

The present invention relates to providing an intervention based on detected gait kinematics for therapy, training, gaming, or movement assistance.

### BACKGROUND

Techniques for providing an intervention, such as stimulating human feet with vibration for therapeutic reasons are known in the art (see for example “Subsensory vibrations to the feet reduce gait variability in elderly fallers”, Galica et al).

Typically, these techniques involve monitoring certain aspects of a subject's gait kinematics to recognize movement requiring vibrating stimulation to be applied, and then applying the relevant vibration.

Often these techniques are used in a motion analysis laboratory. However, certain systems have been proposed that can be used outside of a laboratory setting.

WO2017/023864 for example proposes a system for alleviating knee osteoarthritis by modifying a subject's gait kinematics using electrical or vibrotactile sensory stimulation applied to the subject's foot.

### SUMMARY OF THE INVENTION

According to a first aspect of the invention there is provided a system for providing an intervention based on detected gait kinematics for therapy, training, gaming or movement assistance, said system comprising: at least one item of footwear incorporating one or more sensors, a data processor, and a wireless communication unit; and a remote intervention system configured to provide an intervention for provoking a response by a subject wearing the item of footwear, wherein said one or more sensors is configured to generate sensor data associated with movement of the subject; said data processor is configured to process the sensor data to generate gait parameter data associated with the subject's gait kinematics, and said wireless communication unit is configured to communicate the gait parameter data to the remote intervention system.

Optionally, the remote intervention system is configured to provide a sensory intervention.

Optionally, the remote intervention system comprises at least one stimulation device, such as a spine stimulation device or a deep brain stimulation device or a muscle stimulation device.

Optionally, the remote intervention system comprises a simulator, such as a virtual reality system or an augmented reality system.

Optionally, the at least one item of footwear further comprising a memory, wherein the memory is configured to store sensor data and/or gait parameter data.

Optionally, the data processor is configured to compare sensor data and/or gait parameter data against stored sensor data and/or gait parameter data in order to determine whether the sensor data and/or gait parameter data corresponds to a gait event.

Optionally, the data processor is configured to control the wireless communication unit to communicate gait parameter data to the remote intervention system when it is determined that the sensor data and/or gait parameter data corresponds to a gait event.

Optionally, the gait event is at least one of: an imminent fall or higher risk of fall for the subject, an imminent gait freeze or higher risk of gait freeze, a deviation from a desired movement for the subject, and maintenance of a desired movement form for the subject.

Optionally, the data processor is configured to periodically generate gait parameter data at a predetermined interval and to store the generated gait parameter data in the memory. The predetermined interval may an interval that is suitable for the gait parameter being determined. For example, gait parameter data that can be determined without completion of a complete strep/stride (e.g. gait-stability) may be generated periodically at a predetermined interval that is less than a human reaction time. For example, the interval may be less than 0.1 second. Gait parameter data that requires completion of a complete step/stride (e.g. stride-length) may be generated periodically at a predetermined interval that is not less than the duration of a subject's step/stride (cadence), or a predetermined interval that is not less than the duration of a typical step/stride for a human performing a particular movement (a human locomotion cadence), such as a predetermined interval of not less than 0.5 second and not greater than 2 seconds, such as 1 second.

Optionally, the gait parameter data includes data relating to one or more of gait speed, step/stride velocity, step/stride length, swing time variability, stride length, stride duration, step/stride width, rhythm, variability, asymmetry, postural control, step characteristics, cadence, gait velocity, swing-stance-ratio, heel-off, toe-off, heel-strike, foot-flat-event, gait variability and gait-stability.

Optionally, the one or more sensors, data processor, memory and wireless communication unit are embedded in a sole or an insole of the item of footwear.

Optionally, the sensors comprise one or more inertial measurement units comprising one or more of an accelerometer, gyroscope, and magnetometer.

Optionally, the sensors further comprise one or more of a foot-pressure sensor for detecting pressure changes arising due to the subject contacting the ground, a temperature sensor for detecting an ambient temperature, a barometric pressure senor for detecting barometric pressure and a sound sensor.

Optionally, the at least one item of footwear further incorporates movement distance tracking means configured to generate movement distance data associated with a distance moved by the item of footwear, and said data processor is configured to process the movement distance data to generate movement distance analysis data.

Optionally, the wireless communication unit is configured to communicate the movement distance analysis data to the remote intervention system.

Optionally, the at least one item of footwear comprises a rechargeable battery for powering the components incorporated therein.

According to a second aspect of the invention there is provided a method of providing an intervention based on detected gait kinematics for therapy, training, gaming or movement assistance, said method comprising: generating at an item of footwear sensor data associated with movement of a subject wearing the item of footwear; processing at the item of footwear sensor data to generate gait parameter data associated with the subject's gait kinematics; communicating the gait parameter data from the item of footwear to a remote intervention system for providing an intervention, and controlling the remote intervention system to provide an intervention for provoking a response by a subject wearing the item of footwear.

According to a third aspect of the invention there is provided an arrangement for fitting to an item of footwear, said arrangement comprising one or more sensors, a data processor, and a wireless communication unit, wherein said one or more sensors is configured to generate sensor data associated with movement of a subject wearing the item of footwear; said data processor is configured to process the sensor data to generate gait parameter data associated with the subject's gait kinematics, and said wireless communication unit is configured to communicate the gait parameter data to a remote intervention system.

According to a fourth aspect of the invention there is provided an item of footwear fitted to which is an arrangement according to the third aspect of the invention.

According to a fifth aspect of the invention there is provided a pair of items of footwear, comprising a left hand item of footwear according to the fourth aspect of the invention and a right hand item of footwear according to the fourth aspect of the invention.

According to a sixth aspect of the invention there is provided a computer program for running on a data processor incorporated in an item of footwear and for use in a system according to the first aspect of the invention, said computer program comprising instructions which when implemented on a data processor, controls the data processor to perform a method comprising: generating at an item of footwear sensor data associated with movement of a subject wearing the item of footwear; processing at the item of footwear sensor data to generate gait parameter data associated with the subject's gait kinematics; and communicating the gait parameter data from the item of footwear to a remote intervention system for providing an intervention for provoking a response by a subject wearing the item of footwear.

In accordance with embodiments of the invention a system for providing an intervention based on detected gait kinematics is provided for the purpose of therapy, training, gaming or movement assistance which has an optimized system architecture.

In accordance with embodiments of the invention, data relating to a subject can be collected outside the clinical environment, for example in a familiar setting with unbiased conditions which is likely to lead to intrinsically better analyses and associated therapies. In accordance with embodiments of the invention, a subject's gait can be analyzed quantitatively, objectively and in a reproducible manner. In certain applications it is possible for a therapist, for example, to determine in an objective and unbiased way, if a patient has progressed.

Various further features and aspects of the invention are defined in the claims.

### BRIEF DESCRIPTION OF THE DRAWINGS

Embodiments of the present invention will now be described by way of example only with reference to the accompanying drawings where like parts are provided with corresponding reference numerals and in which:

FIG. 1a provides a simplified schematic diagram of a system arranged in accordance with certain embodiments of the invention;

FIG. 1b provides a simplified schematic diagram of a sensor module arranged in accordance with certain embodiments of the invention;

FIG. 2 provides a flow chart depicting the operation of the system shown in FIG. 1a;

FIG. 3 provides a simplified schematic diagram of the components of a sensor module for fitting to an item of footwear in accordance with certain embodiments of the invention;

FIG. 4 provides a simplified schematic diagram depicting the sensors of a sensor unit in accordance with certain embodiments of the invention;

FIG. 5 provides a simplified schematic diagram depicting the sensors of a further sensor unit in accordance with certain embodiments of the invention;

FIG. 6 provides a diagram depicting the location of vibration actuators in accordance with certain embodiments of the invention;

FIG. 7 provides a simplified schematic diagram depicting the incorporation of a sensor module in a modified shoe sole in accordance with certain embodiments of the invention;

FIG. 8 provides a simplified schematic diagram of the components of a further sensor module for fitting to an item of footwear in accordance with certain embodiments of the invention, in particular further including a location tracking device, and

FIG. 9a provides a simplified schematic diagram depicting an embodiment of the invention in which a sensor module is incorporated in an item of footwear, and

FIG. 9b provides a simplified schematic diagram depicting an embodiment of the invention in which a sensor module is incorporated in an item of footwear.

### DETAILED DESCRIPTION

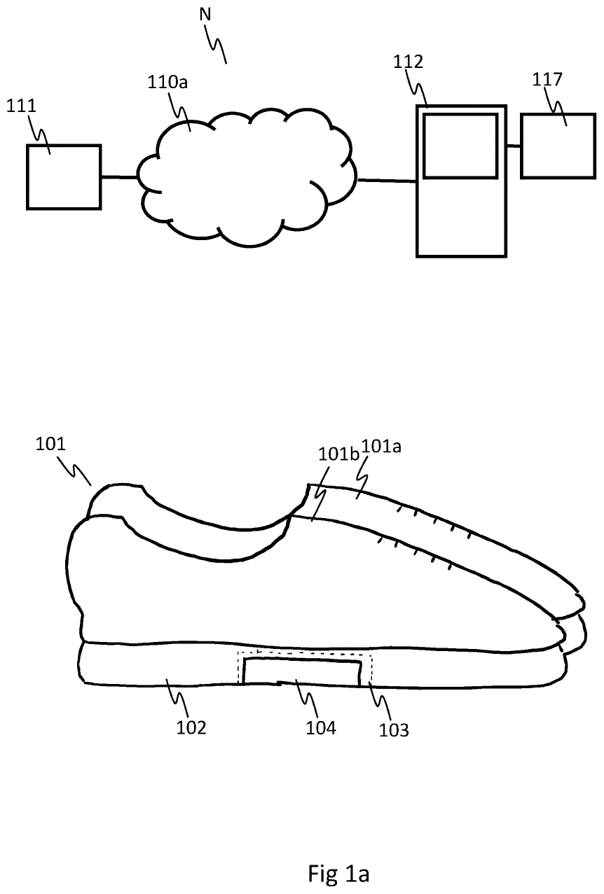

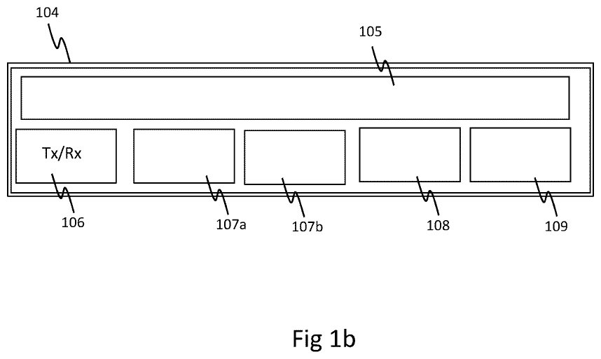

FIGS. 1a provides a schematic diagram of a system for providing an intervention based on detected gait kinematics which comprises a pair of items of footwear 101, and a network arrangement N.

The footwear 101 comprises a pair of items of footwear provided by a pair of shoes 101 comprising a first shoe 101a and a second shoe 101b. Typically, the first shoe 101a and second shoe 101b, other than being configured to fit on the subject's right and left foot respectively, are otherwise identical.

The sole 102 of each shoe 101a, 101b, comprises a cavity 103 within which is mounted a sensor module 104.

As shown in FIG. 1b, the sensor module 104 comprises a power supply unit 105, a wireless communication unit 106, a data processor 107a and corresponding memory unit 107b, an optional vibration actuator 108 and a sensor unit 109 comprising a plurality of sensors.

The power supply unit 105 may be provided by a suitable rechargeable battery as is known in the art. The battery may be recharged by any suitable means, for example by a suitable power cable input interface or by inductive coils incorporated in the power supply for wireless charging.

The network arrangement N comprises a data network 110a, a wireless base station 111 via which the sensor module 104 is configured to transmit data to, and optionally receive data from, and a remote intervention system 112.

The remote intervention system 112 may comprise at least one electrical stimulation device, such as a deep brain stimulation device or a spine stimulation device, that is configured to stimulate a part of the subject which is remote from the region of the foot.

Alternatively, or in addition, the remote intervention system 112 may comprise a simulator, such as a virtual reality system, such as a system providing a metaverse, and/or an augmented reality system, having a screen on which video of the subject and/or imagery of a simulation of the subject can be displayed. The imagery can then be augmented with visuals such as visuals of a metaverse or graphics providing visual cues to the subject.

Generally, an intervention is to be considered in the context of the claimed invention to be any form of feedback to a subject wearing the footwear 101 or a third party, such as a clinician, in order to provoke a response by the subject to the intervention in a desired way. An intervention may be a sensory intervention, such as: an electrical stimulation, a haptic intervention, a visual intervention, such as imagery on a display screen, or an audible intervention amongst others. In the context of the described embodiment, a remote intervention is an intervention which is remote from the region of the foot or feet of a subject on which the footwear is worn.

In certain examples, the data network 110a can be provided by any suitable network for transmitting data between computing devices, for example the internet. The wireless base station 111 can be provided by any suitable wireless access point, that is compatible with the wireless communication unit 106, and is suitable for enabling data to be communicated to and received from the data network 110a for example a suitably connected Wi-Fi router. In alternative embodiments, the wireless base station 111 could be provided by a smart phone, a similar mobile device, a tablet, or any other device with the appropriate communication functionality.

In use, for each sensor module 104 in each shoe, the plurality of sensors of the sensor unit 109 are configured to detect the movement of the subject when wearing the shoe and generate corresponding sensor data associated with this movement. Typically, the sensor data comprises at least one or more of linear acceleration data (generated by an accelerometer), angular velocity data (generated by a gyroscope) and orientation data (generated by a magnetometer).

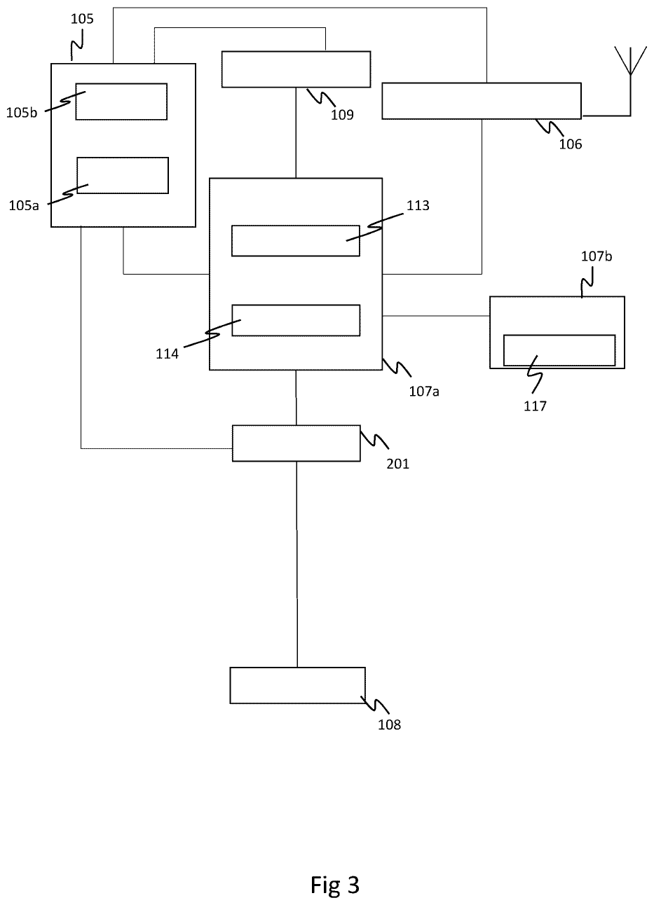

The data processor 107a processes this sensor data to generate gait parameter data associated with the subject's gait kinematics. For example, the data processor 107a has running thereon a gait characterizing function 113, as indicated in FIG. 3, which is configured to process the sensor data from the sensor module of each shoe 101a, 101b to characterize aspects of the subject's gait kinematics.

The gait characterizing function 113 implements one or more gait characterizing algorithms which receive as input the sensor data and, from this, generates gait parameter data associated with the subject's gait as derivable from the sensor data. Techniques for converting such sensor data into gait parameter data are well known. For example, it is well known to use peaks, valleys, and zero/crossings in sensor data generated by sensors monitoring human movement to identify “gait events” such as toe-off and heel-strike and so on.

The gait parameter data generated by the gait characterizing algorithm or gait characterizing algorithms can include data relating to any one of, or any combination of more than one of: gait speed, step velocity, step length, swing time variability, stride length, step width, rhythm (such as step time, swing time, stance time, single support, double support), variability (such as step velocity variability, step length variability, step time variability, stance time variability), asymmetry (such as swing time asymmetry, step time asymmetry, stance time asymmetry), postural control (such as step length asymmetry), step characteristics (strike angle, minimum toe clearance, foot angles (such as supination angle, strike angle, lift-off angle, angular velocity), peak parameters (such as peak propulsion, peak braking), force/pressure values and power. The gait parameters may further include one or more of loading intensity and cycle and pressure distribution.

The gait parameter data is then communicated by the wireless communication unit 106 to the remote intervention system 112 via the wireless base station 111 and data network 110a.

The remote intervention system 112 may be further configured to receive program parameters specified by a program of therapy, program of movement assistance, program of gaming or program of training from a program parameter database 117 connected to the remote intervention system 112. These program parameters quantify how aspects of a subject's gait kinematics will change from their normal movement, in the event that intervention is required.

Using one gait parameter, a combination of gait parameters or all the gait parameters and one or more program parameters specified by a program of therapy, program of movement assistance, program of gaming or program of training, the remote intervention system 112 is configured to determine an appropriate intervention in accordance with the parameters specified by a program of therapy, program of movement assistance, program of gaming or program of training.

For example, where the remote intervention system 112 comprises an electrical stimulation device, such as a deep brain stimulation device or a spine stimulation device, the intervention system 112 applies an appropriate stimulation to the subject which causes the subject to respond in a desired way.

Alternatively, or in addition, the data processor 107a is configured to determine whether or not the determined gait parameter is indicative of a gait event. For example, the data processor 107a may have running thereon a gait event prediction function 114, as indicated in FIG. 3, which is configured to determine that the determined gait parameter corresponds to a gait event such as an imminent fall, gait freeze or deviation from a desired movement.

If a gait event is determined, the data processor 107a controls the wireless communication unit 106 to communicate the gait parameter data which is associated with the gait event to the remote intervention system 112.

For example, the gait event prediction function 114 may determine moving-average data or moving-variance data for one or more gait parameters from gait parameter data stored in the memory unit 107b. For instance, the data processor 107a may be configured to periodically generate gait parameter data at a predetermined interval and to store the generated gait parameter data in the memory unit 107b. The predetermined interval may an interval that is suitable for the gait parameter being determined. For example, gait parameter data that can be determined without completion of a complete strep/stride (e.g. gait-stability) may be generated periodically at a predetermined interval that is less than a human reaction time. For example, the interval may be less than 0.1 second. Gait parameter data that requires completion of a complete step/stride (e.g. stride-length) may be generated periodically at a predetermined interval that is not less than the duration of a subject's step/stride (cadence), or a predetermined interval that is not less than the duration of a typical step/stride for a human performing a particular movement (a human locomotion cadence), such as a predetermined interval of not less than 0.5 second and not greater than 2 seconds, such as 1 second.

A determined gait parameter can then be compared against the moving-average or moving-variance to determine whether a gait event has occurred. If a gait event has occurred, gait parameter data that corresponds to the gait event is communicated to the remote intervention system 112 in order to provide an appropriate intervention.

In addition, the data processor 107a may control the vibration actuator 108 to provide an appropriate stimulation to be applied to the subject's foot based on the determined gait parameter. It will be appreciated that where an intervention, particularly an intervention in the form of a stimulation, is provided by the remote intervention system 112 any stimulation by the vibration actuator 108 may be unnecessary. Typically, vibrations are transferred to the subject's foot via an intermediate portion of the sole separating the vibration actuator 108 and the subject's foot.

In one example the system can be used in a movement assistance program to generate appropriate alert signals to reduce the likelihood that elderly or otherwise vulnerable subjects fall over.

During use, the subject walks around wearing the shoes and corresponding sensor data is generated by the sensors.

The gait characterizing function 113 uses this sensor data to generate gait parameter data relating to the timing of the subject's gait swing when walking (that is, the period of time taken for the subject to complete a stride).

A subject's swing time can typically be determined reliably with simple algorithms. The swing time parameter can be generated by the gait characterizing function 113 identifying from the sensor data the time delay between “toe-off” and “heel-strike” events for each foot.

Specifically, from the sensor data, the gait characterizing function 113 is configured to detect toe-off and heel-strike events by applying a peak-detection algorithm to sensor data associated with the ankle's angular movement rate.

Sensor data associated with a step typically features two peaks, each in proximity of toe-off and heel-strike. Combining this information with sensor data associated with the vertical acceleration (lift-off at toe-off and impact at heel-strike) enables real-time estimates of toe-off and heel-strike events to be generated.

The gait characterizing function 113 is configured to generate swing time data (i.e. gait parameter data). The swing time data is then communicated by the wireless communication unit 106 to the remote intervention system 112.

In response to the received swing data, the remote intervention system 112 provides an appropriate intervention. For example, the remote intervention system 112 may compare the received swing time data against swing time data stored in the program parameter database 117, such as swing time data corresponding to normal movement of a subject, and determine that a fall is imminent.

Where the remote intervention system 112 comprises an electrical stimulation device, such as a deep brain stimulation device or a spine stimulation device, the remote intervention system 112 then generates a sensory stimulation to alert the subject that they might be about to fall and/or stimulates the subject's body to make appropriate movements to prevent a fall.

Alternatively, or in addition, the gait characterizing function 113 is configured to identify from the sensor data a number of toe-off and heel-strike events and generate a plurality of swing time values.

The gait characterizing function 113 is then configured to generate, from this plurality of swing time values, an average swing time value which is an average of the plurality of swing time values.

The gait characterizing function 113 is further configured to calculate, using the plurality of swing time values, a time value corresponding to one standard deviation from this average swing time value of the plurality of swing time values. The plurality of swing time values, average swing time value and standard deviation of the swing time value may be stored in the memory unit 107b.

The data processor 107a has running thereon a gait event prediction function 114 which is configured to process the swing time gait parameter data generated by the gait characterizing function 113 and to determine whether the gait parameter data is indicative of an imminent fall. For example, gait event prediction function 114 may be configured to determine whether the determined swing time data is within a predetermined number of standard deviations from a subject's average swing time by which the subject's swing time will increase if a fall is predicted to be imminent.

For example, the gait characterizing function 113 may calculate from the sensor data that the subject's average swing time is 700 ms with a distribution of swing times such that one standard deviation from the average swing time is 250 ms. The gait parameter data communicated from the gait characterizing function 113 to the gait event prediction function 114 will therefore specify an average swing time value of 700 ms and a standard deviation time value corresponding to one standard deviation of 250 ms.

Further, the program parameter database 117 may comprise program parameter data specifying that if a threshold increase in a subject's swing time of two deviations from the average swing time is met or exceeded, this is indicative of an imminent fall.

Thus in this example, this will be a gait swing time of at least:

700 ms+(2×250 ms)=1200 ms

In such an example, the gait event prediction function 114 is configured to determine that a fall is imminent if the subject's swing time is 1200 ms or greater.

Therefore, in the event that the subject's gait swing time changes to exceed 1200 ms, the data processor 107a identifies that the determined swing time exceeds the threshold gait swing time value and communicates the swing time (i.e. gait parameter data) to the remote intervention system 112 via the wireless communication unit 106.

In response to the received swing time data, the remote intervention system 112 provides an appropriate intervention. For example, where the remote intervention system 112 comprises an electrical stimulation device, such as a deep brain stimulation device or a spine stimulation device, the remote intervention system 112 generates a sensory stimulation to alert the subject that they might be about to fall and/or stimulates the subject's body to make appropriate movements to prevent a fall.

The data processor 107a may also generate a corresponding control signal which when received by the vibration actuator 108, causes the vibration actuator 108 to generate a corresponding sensory stimulation to alert the subject that they might be about to fall. The subject, thus alerted, may then be less likely to fall.

The detection of sensor data associated with swing time by the data processor 107a typically occurs at a rate higher than customary human reaction times. In this way, the subject experiences smooth and “instantaneous” feedback, as soon as the set threshold is passed, and the intervention is triggered. Typically, human reaction times are around 0.1 s, detecting the subject's gait swing time at 100 Hz will result in smooth operation.

In another example, the system can be used in a program of therapy to assist subjects suffering from neurological disorders such as multiple sclerosis or Parkinson's disease and who experience “gait freeze”.

In such an example, in keeping with the previous example, the subject walks around wearing the shoes and corresponding sensor data is generated by the sensors.

The gait characterizing function 113 uses this sensor data to generate gait parameter data including swing time gait parameter associated with the subject's gait swing time when walking normally, as per the previous example.

The gait characterizing function 113 is configured to generate swing time data (i.e. gait parameter data). The swing time data is then communicated by the wireless communication unit 106 to the remote intervention system 112.

In response to the received swing data, the remote intervention system 112 provides an appropriate intervention. For example, the remote intervention system 112 may compare the received swing time data against swing time data stored in the program parameter database 117, such as swing time data correlating a reduction in average swing time of more than a predetermined amount (e.g. 50%) over a predetermined period (e.g. 60 seconds) to an impending gait freeze, and determine an impending gait freeze.

Where the remote intervention system 112 comprises an electrical stimulation device, such as a deep brain stimulation device or a spine stimulation device, the remote intervention system 112 then generates a sensory stimulation to alert the subject that they might be about to fall and/or stimulates the subject's body to make appropriate movements to prevent an impending gait freeze.

Alternatively, or in addition, the memory unit 107b may have stored therein program parameters which specify a reduction in average swing time of more than a predetermined amount (e.g. 50%) over a predetermined period (e.g. 60 seconds) is indicative of an impending gait freeze. In the event that the subject's average swing time reduces by at least the threshold amount (e.g. at least 50%) during the threshold period of time (e.g. within 60 seconds) indicating an impending episode of gait freeze, the gait event prediction function 114 generates “gait freeze” imminent gait parameter event data which is communicated to the remote intervention system 112.

In response to the received “gait freeze” imminent gait parameter event data, the intervention system 112 provides an appropriate intervention. For example, where the intervention system 112 comprises an electrical stimulation device, such as a deep brain stimulation device or a spine stimulation device, the intervention system 112 generates a sensory stimulation to alert the subject that they might be about to suffer from an episode of gait freeze and/or to stimulate the subject's body to make appropriate movements to prevent an episode of gait freeze.

The data processor 107a may also generate a corresponding control signal which when received by the vibration actuator 108, which causes the vibration actuator 108 to generate a corresponding sensory stimulation to alert the subject that initiate a step and end the gait freeze. The subject, thus alerted, may then be less likely to suffer from an episode of gait freeze.

In another example, the system can be used in a program of training to assist a subject seeking to improve their technique when performing an activity such as running.

In such an example, in keeping with the previous example, the subject moves around wearing the shoes, and in particular moves around with a particular desired movement form. Corresponding sensor data is generated by the sensors.

The gait characterizing function 113 uses this sensor data to generate gait parameter data indicative of one or more gait parameters associated with the desired movement form, for example step width and swing time.

The gait characterizing function 113 is configured to generate swing time data and step width data (i.e. gait parameter data). The swing time data and step width data is then communicated via the wireless communication unit 106 to the remote intervention system 112.

In response to the received swing time data and step width data, the remote intervention system 112 provides an appropriate intervention. For example, the remote intervention system 112 may compare the received swing time data and step width data against swing time data and step width data stored in the program parameter database 117, in order to identify variations in swing time and step width of more than 5% relative to those specified by a movement training program stored in the program parameter database 117.

Where the remote intervention system 112 comprises an electrical stimulation device, such as a deep brain stimulation device, a spine stimulation device or a muscle stimulation device, the remote intervention system 112 then generates a sensory stimulation to alert the subject the subject's movements deviated from a desired form and/or to stimulate the subject's body to make appropriate movements to correct the deviation.

Alternatively, or in addition, the memory unit 107b may have stored therein program parameters associated with a movement training program which specify swing time data and step width data parameters against which received swing time data and step width data is compared. In the event that the subject's received swing time data and step width data vary by more than 5% relative to those specified by the movement training program, indicating a deviation from a desired movement form, the gait event prediction function 114 generates form deviation gait event data which is communicated to the remote intervention system 112.

In response to the received form deviation gait event data, the intervention system 112 provides an appropriate intervention. For example, where the intervention system 112 comprises an electrical stimulation device, such as a deep brain stimulation device or a spine stimulation device, the intervention system 112 generates a sensory stimulation to alert the subject that they have deviated from a desired form and/or to stimulate the subject's body to make appropriate corrective movements.

The data processor 107a may also generate a corresponding control signal which when received by the vibration actuator 108, which causes the vibration actuator 108 to generate a corresponding sensory stimulation to alert the subject to make appropriate corrective movements.

However, in certain examples, communication of the form deviation gait event to the remote intervention system 112 causes the remote intervention system 112 to cease the application of an intervention. For example, the remote intervention system 112 may be configured, in accordance with a program of training to provide a regular intervention which conveys to a subject (such as an athlete) that they are moving at a desired pace. Upon determination that the subject has dropped below or exceeded this pace, corresponding pace discrepancy gait event data is generated and communicated to the remote intervention system 112 which then ceases to provide the regular intervention until the desired pace is achieved again.

As a further example, the remote intervention system 112 may comprise an augmented reality (AR) system in which imagery of the real world environment is augmented by computer-generated sensory stimuli (e.g. a visual stimulation such as an image, an audible stimulation or a haptic stimulation) or a virtual reality (VR) system in which a virtual world is simulated. An AR system may include virtual objects which are spacially registered as objects within the imagery of the real world environment. A virtual reality (VR) system may include a simulation within which the subject is represented such as a metaverse. In this context, an intervention provided by a remote intervention system comprising an AR/VR system is any sensory stimulation that can be experienced by a person, particularly the subject.

In the particular context of a system which can be used in a program of training to assist a subject seeking to improve their technique when performing an activity such as running, the remote intervention system 112 comprises augmented reality (AR) system or virtual reality (VR) system.

The gait characterizing function 113 uses sensor data obtained while a subject moves around using the shoes to generate gait parameter data indicative of one or more gait parameters associated with the desired movement form.

The gait parameter data is then communicated by the data processor 107a via the wireless communication unit 106 to the remote intervention system 112. The gait parameter data is then processed by the remote intervention system 112 to provide an intervention in the form of a sensory feedback, such as a visual feedback, to which the subject then responds. For example, the visual feedback may be a virtual target which appears in front of the subject (or a representation of the subject) on a display of the AR/VR system and which the subject is instructed to follow. If the subject's pace is determined to be too slow, the virtual target can be moved further away from the subject in the AR/VR system thereby stimulating the subject to increase their pace. Alternatively, or in addition, the visual feedback may be a modification of the virtual environment according to a predetermined training program. For example, steps taken by a subject may be replicated in a virtual reality system, such as a metaverse.

Program parameters stored in the program parameter database 117 or memory unit 107b are defined based on the type of program being provided (e.g. a training program, a therapy program, gaming program, or a program of movement assistance). In each case the program parameters may be selected based on relevant research. For example, the program parameters for a therapy program attempting to alleviate or reduce the occurrence of gait freeze will be defined in part based on what research into this condition suggests is effective for treating the condition.

Program parameters may also be defined in part by characteristics of the subject who will be using the system. For example, characteristics such as age, weight, sex, height and so on. For example, in programs for providing movement assistance which aim to reduce the likelihood of subjects falling, the variation in swing time that is identified to predict an imminent fall may vary in dependence on the age of the subject.

Program parameters may also be defined in part by historic data associated with the subject. For example, a particular subject may have previously exhibited a certain sequence of gait kinematics ahead of the occurrence of an episode of gait freeze. in such examples, the program parameters may be selected to specify these gait kinematics.

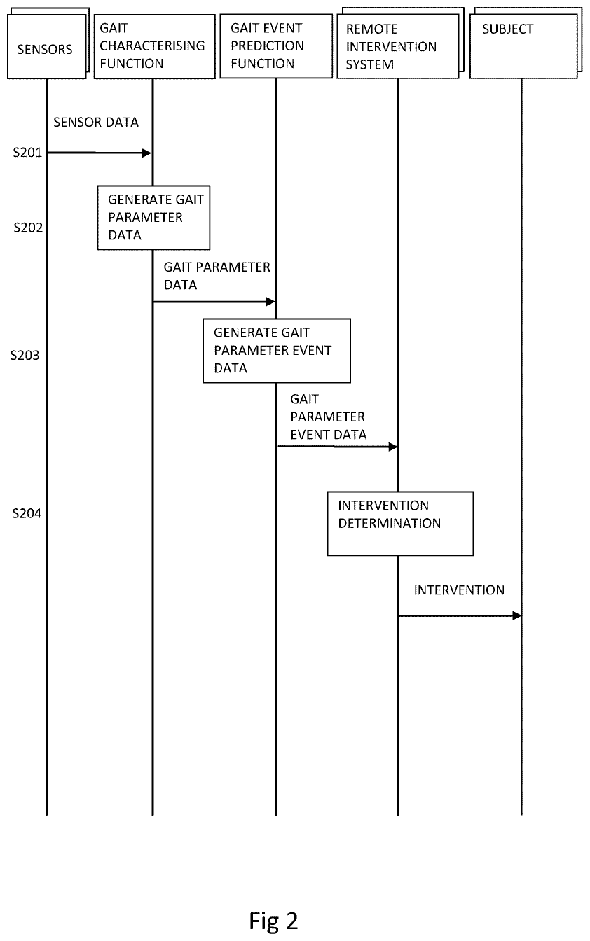

FIG. 2 provides a schematic diagram summarizing one mode of operation of the system as described above.

At a first stage S201 sensor data is generated by the sensors of at least one of the shoes of the pair of shoes.

At a second stage S202 the gait characterizing function 113 processes the sensor data to generate gait parameter data associated with the subject's gait kinematics.

At a third stage S203 the gait event prediction function 114 determines whether the gait parameter data is indicative of a gait event and, if the gait parameter data is indicative of a gait event, the gait parameter data is communicated to the remote intervention system.

At a fourth stage S204, responsive to receipt of the gait parameter data, the remote intervention system generates an intervention to which the subject responds.

Advantageously, this means that the training program, movement assistance program or therapy program can continue to be effectively adapted as the subject's gait kinematics change over time, for example, due to changes brought about by the training program or therapy program.

FIG. 3 provides a schematic diagram providing a more detailed view of a sensor module 104 arranged in accordance with certain embodiments of the invention.

As can be seen, the sensor module 104 comprises a power supply unit 105 which, in certain embodiments comprises one or more rechargeable batteries 105a and an inductive charging loop 105b for charging the rechargeable batteries 105a via a wireless charging unit.

The sensor module 104 further comprises the data processor 107a which can be provided by any suitable programmable microprocessor or by other appropriate data processing means, for example a custom-designed integrated circuit such as a field programmable gate array (FPGA).

The data processor 107a is connected to a motor power control circuit via a suitable signal line which is connected, via a further suitable signal line, to the vibration actuator 108.

The vibration actuator is typically provided by an electric motor comprising an eccentrically mounted weight on a shaft of the motor. However, the vibration actuator can be provided by other suitable electro-mechanical devices, for example piezoelectric vibration actuators and voice-coil-like linear electromagnetic actuators (“tactors”).

The sensor unit 109 is connected via a suitable signal line to the data processor 107a. The sensor unit 109 is typically provided by an inertial measurement unit (IMU) comprising an accelerometer, gyroscope, and magnetometer, connected to the data processor unit.

The wireless communication unit 106 is also connected to the data processor 107a via a suitable signal line. The wireless communication unit 106 can be provided by any suitable wireless communication unit operating in accordance with conventional radio protocols such as Bluetooth, Zigbee, LoRa, NFC, WiFi and so on. In certain examples the wireless communication unit 106 can be provided by a data transmitter, a receiver and/or a transceiver provided with a subscriber identity module (SIM) and enabling data to be transmitted to and from the data network 110a via a cellular mobile telephone network.

All of the components of the sensor module 104 are connected to the power supply unit 105 via suitable power lines.

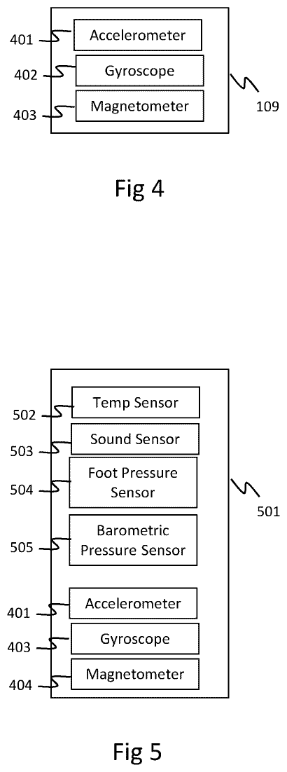

FIG. 4 provides a schematic diagram depicting in more detail the sensors of the sensor unit 109 in accordance with certain embodiments of the invention. As can be seen from FIG. 4, the sensor unit 109 comprises an accelerometer 401, a gyroscope 402 and a magnetometer 403. This combination of sensors typically provides enough information about a subject's movement to enable aspects of the subject's gait to be characterized including gait speed, step velocity, step length, swing time variability, stride length, step width, rhythm (such as step time, swing time, stance time, single support, double support), variability (such as step velocity variability, step length variability, step time variability, stance time variability), asymmetry (such as swing time asymmetry, step time asymmetry, stance time asymmetry), postural control (such as step length asymmetry), step characteristics (strike angle, minimum toe clearance, foot angles (such as supination angle, strike angle, lift-off angle, angular velocity) and peak parameters such as peak propulsion and peak braking.

In certain embodiments, the sensor unit 109 may comprise one or more further sensors.

FIG. 5 provides a schematic diagram depicting a further example of a sensor unit 501 comprising the sensors of the sensor unit 109 shown in FIG. 4 comprising further sensors, specifically, a temperature sensor 502, a sound sensor 503, a foot pressure sensor 504, and a barometric pressure sensor 505.

In use, the temperature sensor 502 is configured to measure the temperature and generate corresponding temperature data. This temperature data is communicated to the data processor 107a. The data processor 107a is configured to use this temperature data to calibrate, if needed, sensor data from the sensor unit 109 to account for changes (drift) in the output of the sensor unit 109 arising due to changes in temperature to which the system is exposed.

In certain examples, the data processor 107a is configured to process the sensor data generated by the sound sensor 503, the foot pressure sensor 504, and the barometric pressure sensor 505 in order to generate gait parameter data.

In use, the foot-pressure sensor 504 is typically positioned so that pressure changes arising due to the subject contacting the ground can be detected. For example, the foot-pressure sensor 504 can be provided by a two-dimensional pressure sensing pad configured to be positioned across the base, or part of the base, of a modified shoe sole so that the pressure at different contact points of the subject's foot as it contacts the ground can be measured. The foot-pressure sensor 504 is configured to generate pressure data. The gait characterizing function 113 is configured to use the pressure data when generating the gait parameters. For example, the gait characterizing function 113 can use the pressure data to determine points in time when the subject's foot is in contact with the ground and/or using it to determine points in time when particular regions of the subject's foot (for example the ball and the heel) are in contact with the ground. Further information relevant to analyzing a subject's gait can be determined from the pressure data such as the impact force with which a subject is contacting the ground with their foot or specific regions of their foot.

In use, the sound sensor 503 is configured to detect sound in the region of the subject's foot and to generate corresponding sound data. In certain embodiments, the gait characterizing function is configured to use the sound data to classify the type of surface that the subject is moving (walking, running) on which can then be used to refine the algorithms used to estimate the subject's gait parameters. In certain examples, the sound data can be used to detect the type or place of the subject's activity.

In use, the barometric pressure sensor 505 is configured to detect the atmospheric pressure around the shoe and generate corresponding barometric pressure sensor data. This barometric pressure sensor data may be used by an altitude detection function of the data processor 107a which is configured to receive the sensor data from the barometric pressure sensor to generate corresponding altitude data. This altitude data can be used to track vertical movement of the subject whilst wearing the shoes, for example as part of an exercise program.

In certain embodiments the altitude detection function can be incorporated in a movement distance analysis function as described in more detail below.

The skilled person will understand that the arrangement of the components of the system is one example of how a system in accordance with embodiments of the invention can be arranged and the components of the system can be manifested in any suitable alternative way.

For example, in other configurations the intervention system may comprise a personal computing device such as a personal computer (“PC”), tablet computer, smartphone or similar and the program parameters stored on such a device in a suitable memory.

In certain embodiments, each sensor module and the remote intervention system may communicate directly via a suitable data link, that is, without an intermediate data network and/or without an intermediate base station as is shown in FIG. 1a. For example, the remote intervention system and each sensor module may communicate data with each other via a short-range radio protocol such as Bluetooth, WiFi or similar.

In embodiments of the invention, the sensor module can be configured (by virtue of the positioning of the vibration actuator with respect to the sole of the item of footwear) to stimulate any suitable region on the underside (inferior) side of a subject's foot (the sole of the foot).

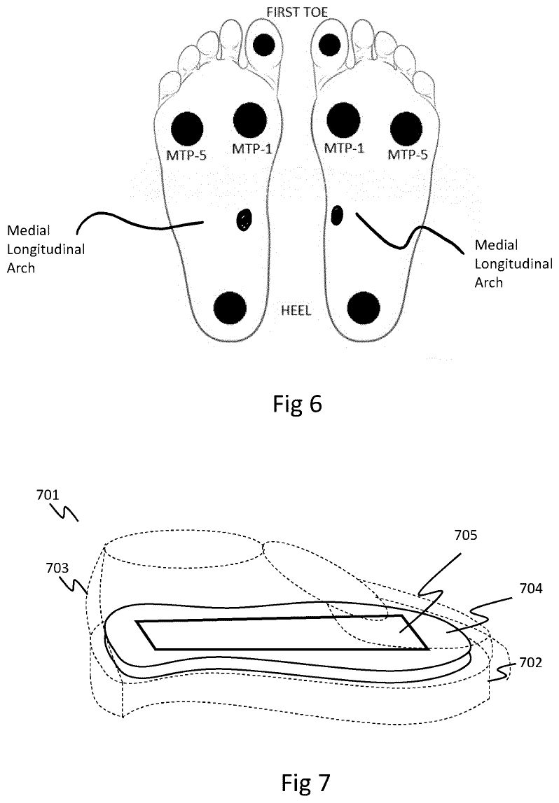

These regions include the first metatarsophalangeal joint; the fifth metatarsophalangeal joint, region of the heel; the region of the big toe and the medial longitudinal arch. These example regions are shown in FIG. 6. In examples in which the item of footwear incorporates a single vibration actuator, that actuator may be positioned at any one of the positions, although the skilled person will recognize that other locations not shown in FIG. 6 are possible.

In certain examples, each item of footwear may be provided with more than one vibration actuator. In such examples, the sensor module can be configured such that the vibration actuators are positioned to provide sensory stimulation to any suitable combination of regions of the underside of the subject's foot. For example, a first vibration actuator can be positioned so as to stimulate a foot position in the region of the first metatarsophalangeal joint; a second vibration actuator can be positioned so as to stimulate a foot position in the region of the fifth metatarsophalangeal joint, and a third vibration actuator can be positioned in the so as to stimulate a foot position in the region of the heel. In certain other embodiments, a fourth vibration actuator can be positioned so as to stimulate a foot position in the region of the big toe.

In certain embodiments the number of vibration actuators and the position of vibration actuators will be selected based on the type of therapy or training being delivered to the subject because, for example, stimulation in different locations can induce different reactions in different patient groups.

In the examples above, the foot stimulation vibration can be “sensory” so that the subject is consciously aware of the vibration. This is an example of “tactile cuing” whereby the subject receives “cues” via consciously detectable tactile stimulation.

However, in certain examples, for example where vibration is being applied as therapy for example for diabetic neuropathy patients, or to prevent falls or in response to foot freeze, the vibration can be sub-sensory such that the subject may not be consciously aware of the vibration, but the vibration nevertheless generates neurological stimulation and produces a desired effect such as improving balance and walking. In such examples, the foot stimulating vibration generated by the vibration actuator or vibration actuators is subsensory vibration (not consciously detectable by the subject).

Particularly in examples in which subsensory vibrations are generated, the data processor associated with each item of footwear can be configured to calibrate the vibration generated by the vibration actuator (or each vibration actuator) to take account of the fact that different subjects have different sensory threshold levels, and these sensory thresholds will vary across different areas of a subject's foot.

To facilitate this, the data processor associated with each item of footwear can be configured to implement a calibration process which controls the vibration actuator (or each vibration actuator) to iteratively step through a sequence of different vibration levels until a vibration level is identified, that is just below a subject's sensory perception for the region of the subject's foot that the vibration actuator stimulates. This calibration process can be carried out in conjunction with an external device, for example a mobile computing device, such as a smart phone, connected to the data processor via the data transceiver and a suitable wireless link.

Different vibration levels can be provided by the vibration actuators vibrating at different frequencies (for example when the vibration actuators are provided by electric motors comprising an eccentrically mounted weight on a shaft of the motor) and/or the vibration actuators vibrating at different amplitudes (for example where the vibration actuators are provided by voice-coil-like linear electromagnetic actuators (“tactors”)).

In this way, after the calibration process is completed, a vibration level (typically consisting of a vibration frequency and/or vibration amplitude) will be determined for each vibration actuator which is then used during operation of the system.

In certain examples, the data processor in each item of footwear controls the vibration actuator (or each vibration actuator) to generate the foot stimulating vibration using “stochastic resonance”. In such examples, the foot stimulating vibration is generated in accordance with a random pattern (which is typically more effective for neuro-stimulation). For example, the vibration actuators can be configured to apply the foot stimulating vibration in an “on/off” pattern, with the time between the “on” and “off” phases varying randomly between, for example, 0.01 s and 0.09 s.

In certain examples, sensory stimulating apparatus in accordance with embodiments of the invention can be incorporated in a modified insole which can be inserted and removed from an item of footwear. An example of such an embodiment is depicted in FIG. 7. FIG. 7 provides a simplified schematic diagram showing an otherwise conventional item of footwear 701 comprising a sole 702 and an upper 703 (the sole 702 and upper 703 are indicated with broken lines and shown transparently). A removable modified insole 704 is shown incorporated within which is an assembly 705 comprising a vibration generating apparatus.

As will be understood, the removable modified insole 704 can be removed from the item of footwear 701 and placed in a different item of footwear. This allows, for example, the modified insole 704 to be used in the footwear of multiple subjects or multiple items of footwear of the same subject. The modified insole 704 may comprise a washable and/or otherwise disinfectable outer which enables the modified insole 704 to be cleaned, for example, for the purposes of hygiene, after use in the footwear of a first subject and before use in the footwear of a second subject.

In the example described with reference to FIG. 1a, all the components associated with detecting the motion of the subject and applying the sensory stimulation are incorporated in a single sensory stimulation unit. However, in other examples these components maybe integrated with the item in footwear in different ways. For example, the vibration actuator or vibration actuators can be fitted to a modified shoe sole or modified insole whilst other components, for example the sensors and the data processor, can be incorporated in other parts of the item of footwear, such as, for example, a shoe upper or a shoe tongue.

Embodiments of the invention can be used with any suitable form of footwear. Such footwear includes shoes such as trainers (sneakers), boots and sandals. In certain embodiments, vibration generating apparatus can be incorporated in specific medical footwear such as controlled ankle motion (CAM) walking boots (“moonboots”).

In certain embodiments the sensor module of one or both shoes are configured to implement a movement distance tracking function. The movement distance tracking function is configured to track the distance the shoe has moved and generate corresponding movement distance data.

The movement distance analysis function may be configured to analyze the movement distance data to determine movement patterns associated with movement of the shoe (for example, total distance moved, average time moving, maximum and minimum movement distances over set periods and so on) and generate corresponding movement distance analysis data. The movement distance analysis data can then be used to optimize the program of therapy, movement assistance, gaming or training being provided by the system. For example, a specialist (such as a physician) can manually change the program parameters stored in the program parameter database based on a pattern of movement distance by the subject.

The movement distance tracking function can be implemented by any suitable means. For example a movement distance tracking function can be implemented on the data processor on the sensor module of one or both shoes and the sensor module can be further equipped with a location tracking device (such as a global navigation satellite system (GNSS) receiver, for example a GPS receiver). The data processor is configured to receive location data from the location tracking device and from this generate movement distance data. In other examples, the movement tracking function can be configured to use the sensor data collected by the sensor unit (for example, inferring a total distance moved by estimating the number of steps taken by the subject) and from this generate movement distance data.

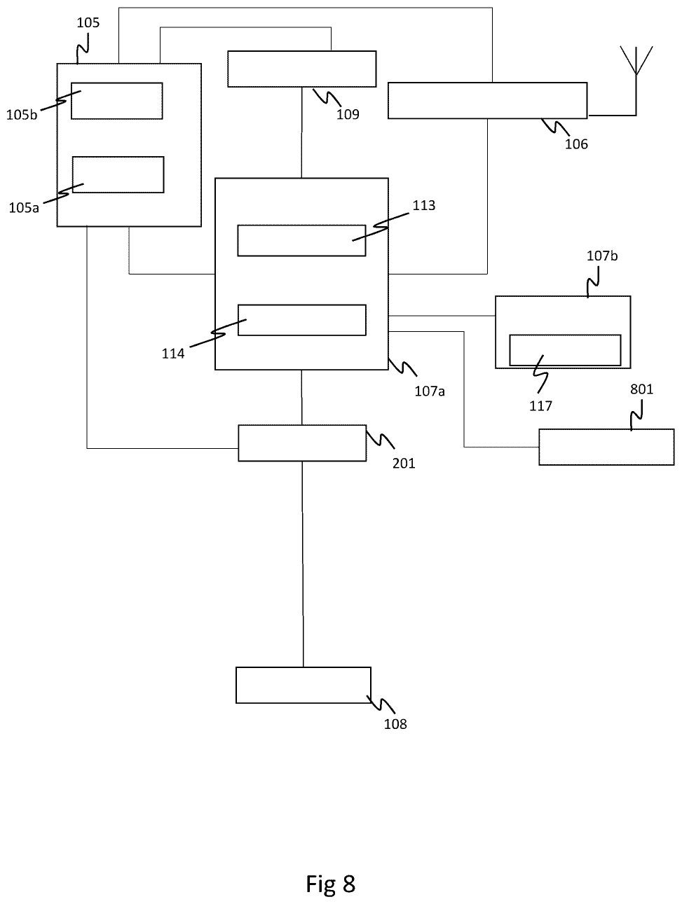

FIG. 8 provides a schematic diagram of a sensor module arranged for this purpose. FIG. 8 shows a sensor module corresponding to the sensor module described with reference to FIG. 3, except that it further comprises a location tracking device 801 provided by a GNSS receiver such as a GPS receiver.

As described above, in certain embodiments the movement distance analysis function can incorporate the altitude detection function so that movement patterns associated with altitude (for example, number of metres ascended and/or descended during a given period of time) can also be taken into account when generating movement distance analysis data.

In certain embodiments, the system is provided with further functionality that enables sensory stimulation to be generated for further purposes.

For example, in certain embodiments, the system is configured to provide tactile cueing to prompt the subject during training or testing.

Such prompting can include prompting the subject to perform actions such as start, stop, turn, sit down, stand up and so on.

Such embodiments can be implemented in any suitable way.

The data processor of the sensor unit has running thereon a tactile cueing generation function.

In this way, for example, a sequence of tactile cueing vibrations could be generated prompting the subject to start walking then stop walking then start walking again and so on during a training or therapy session.

In the example embodiments described above, the vibration actuators of the sensor module are positioned and configured such that sensory stimulation is applied principally to the underside (inferior side) of a subject's foot, that is the sole of a subject's foot.

However, in other embodiments, the sensor module may be configured alternatively or additionally to apply sensory stimulation to other regions of subject's foot. For example, in certain embodiments, an item of footwear is provided incorporating a sensor module substantially corresponding to those described above except that the vibration actuator or vibration actuators of the sensor module are positioned and configured to apply sensory stimulation to the subject's ankle or region immediately adjacent to the subject ankle.

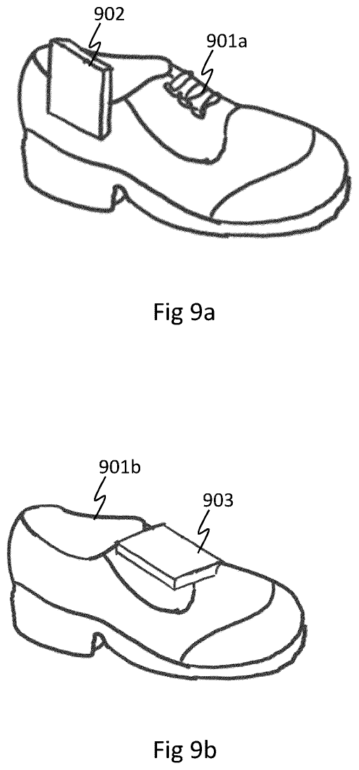

FIG. 9a provides a simplified schematic diagram of such an embodiment. FIG. 9a shows an item of footwear 901a comprising a sensor module 902 of the type described above and including, for example, all the components depicted in FIG. 3. As can be seen from FIG. 9a, the sensor module 902 is mounted to the item of footwear 901 a in a position such that during use sensory stimulation will be applied to the subject's distal ankle.

In further embodiments, an item of footwear is provided incorporating a sensor module substantially corresponding to those described above except that the vibration actuator or vibration actuators of the sensor module are positioned and configured to apply sensory stimulation to an upper (superior) side of the subject's foot.

FIG. 9b provides a simplified schematic diagram of such an embodiment. FIG. 9b shows an item of footwear 901b comprising a sensor module 903 of the type described above and including, for example, all the components depicted in FIG. 3. As can be seen from FIG. 9b, the sensor module 902 is mounted to the item of footwear 901a in a position such that during use, sensory stimulation will be applied to the upper (superior) side of the subject's foot.

All of the features disclosed in this specification (including any accompanying claims, abstract and drawings), and/or all of the steps of any method or process so disclosed, may be combined in any combination, except combinations where at least some of such features and/or steps are mutually exclusive. Each feature disclosed in this specification (including any accompanying claims, abstract and drawings) may be replaced by alternative features serving the same, equivalent, or similar purpose, unless expressly stated otherwise. Thus, unless expressly stated otherwise, each feature disclosed is one example only of a generic series of equivalent or similar features. The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

It will be appreciated that various embodiments of the present disclosure have been described herein for purposes of illustration, and that various modifications may be made without departing from the scope of the present disclosure. Accordingly, the various embodiments disclosed herein are not intended to be limiting, with the true scope being indicated by the following claims.

1. A system for providing an intervention based on detected gait kinematics for therapy, training, gaming or movement assistance, said system comprising:
at least one item of footwear incorporating one or more sensors, a data processor, and a wireless communication unit; and
a remote intervention system configured to provide an intervention for provoking a response by a subject wearing said item of footwear, wherein
said one or more sensors is configured to generate sensor data associated with movement of the subject;
said data processor is configured to process the sensor data to generate gait parameter data associated with the subject's gait kinematics, and
said wireless communication unit is configured to communicate the gait parameter data to the remote intervention system.

2. A system according to claim 1, wherein the remote intervention system is configured to provide a sensory intervention.

3. A system according to claim 1, wherein the remote intervention system comprises at least one stimulation device, such as a spine stimulation device or a deep brain stimulation device or a muscle stimulation device.

4. A system according to claim 1, wherein the remote intervention system comprises a simulator, such as a virtual reality system or an augmented reality system.

5. A system according to claim 1, the at least one item of footwear further comprising a memory, wherein the memory is configured to store sensor data and/or gait parameter data.

6. A system according to claim 5, wherein the data processor is configured to compare sensor data and/or gait parameter data against stored sensor data and/or gait parameter data in order to determine whether the sensor data and/or gait parameter data corresponds to a gait event.

7. A system according to claim 6, wherein the data processor is configured to control the wireless communication unit to communicate gait parameter data to the remote intervention system when it is determined that the sensor data and/or gait parameter data corresponds to a gait event.

8. A system according to claim 6, wherein the gait event is at least one of: an imminent fall or higher risk of fall for the subject, an imminent gait freeze or higher risk of gait freeze, a deviation from a desired movement for the subject, and maintenance of a desired movement form for the subject.

9. A system according to claim 5, wherein the data processor is configured to periodically generate gait parameter data at a predetermined interval and to store the generated gait parameter data in the memory.

10. A system according to claim 1, wherein the gait parameter data includes data relating to one or more of gait speed, step/stride velocity, step/stride length, swing time variability, stride length, stride duration, step/stride width, rhythm, variability, asymmetry, postural control, step characteristics, cadence, gait velocity, swing-stance-ratio, heel-off, toe-off, heel-strike, foot-flat-event, gait variability and gait-stability.

11. A system according to claim 1, wherein the one or more sensors, data processor, memory and wireless communication unit are embedded in a sole or an insole of the item of footwear.

12. A system according to claim 1, wherein the sensors comprise one or more inertial measurement units comprising one or more of an accelerometer, gyroscope, and magnetometer.

13. A system according to claim 12, wherein the sensors further comprise one or more of a foot-pressure sensor for detecting pressure changes arising due to the subject contacting the ground, a temperature sensor for detecting an ambient temperature, a barometric pressure senor for detecting barometric pressure and a sound sensor.

14. A system according to claim 1, wherein the at least one item of footwear further incorporates movement distance tracking means configured to generate movement distance data associated with a distance moved by the item of footwear, and
said data processor is configured to process the movement distance data to generate movement distance analysis data.

15. A system according to claim 14, wherein the wireless communication unit is configured to communicate the movement distance analysis data to the remote intervention system.

16. A system according to claim 1, wherein the at least one item of footwear comprises a rechargeable battery for powering the components incorporated therein.

17. A method of providing an intervention based on detected gait kinematics for therapy, training, gaming or movement assistance, said method comprising:
generating at an item of footwear sensor data associated with movement of a subject wearing the item of footwear;
processing at the item of footwear sensor data to generate gait parameter data associated with the subject's gait kinematics;
communicating the gait parameter data from the item of footwear to a remote intervention system for providing an intervention, and
controlling the remote intervention system to provide an intervention for provoking a response by a subject wearing the item of footwear.

18. An arrangement for fitting to an item of footwear, said arrangement comprising one or more sensors, a data processor, and a wireless communication unit, wherein
said one or more sensors is configured to generate sensor data associated with movement of a subject wearing the item of footwear;
said data processor is configured to process the sensor data to generate gait parameter data associated with the subject's gait kinematics, and
said wireless communication unit is configured to communicate the gait parameter data to a remote intervention system.

19. An item of footwear fitted to which is an arrangement according to claim 18.

20. A pair of items of footwear, comprising a left hand item of footwear according to claim 19 and a right hand item of footwear according to claim 19.

21. A computer program for running on a data processor incorporated in an item of footwear and for use in a system according to claim 1, said computer program comprising instructions which when implemented on a data processor, controls the data processor to perform a method comprising:
generating at an item of footwear sensor data associated with movement of a subject wearing the item of footwear;
processing at the item of footwear sensor data to generate gait parameter data associated with the subject's gait kinematics; and
communicating the gait parameter data from the item of footwear to a remote intervention system for providing an intervention for provoking a response by a subject wearing the item of footwear.
